(19) United States
(12) Patent Application Publication
(10) Pub. No.: US 2005/0112625 A1
(43) Pub. Date: May 26, 2005

(54) DNA amplification method

(75) Inventor: Akihisa Nakajima, Sagamihara-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: KONICA MINOLTA MEDICAL & GRAPHIC, INC.

(21) Appl. No.: 10/920,271

(22) Filed: Aug. 18, 2004

(30) Foreign Application Priority Data
Aug. 22, 2003 (JP) JP2003-299369
Nov. 26, 2003 (JP) JP2003-395640

(51) Int. Cl. C12Q 1/68; C12P 19/34

(52) U.S. Cl. 435/6; 435/91.2

(57) ABSTRACT

A DNA amplification method is disclosed, comprising (a) denaturing a DNA to separate a double-stranded DNA into two single-stranded DNA, (b) hybridizing two types of oligonucleotides which were designed so that the end of the 3′ side of the region of a sequence to be amplified is identified and hybridized with each single-stranded DNA that has been separated, (c) elongating the oligonucleotides with a DNA polymerase in the presence of deoxyribonycleotide triphosphate, and (d) repeating steps (a) through (c) to perform DNA amplification, wherein in step (b), the two types of oligonucleotides are oligonucleotides in which a polymer having a lower critical solution temperature is linked to the 5′ end.

### FIELD OF THE INVENTION

The present invention relates to a method for simpler detection of the presence of a target DNA sequence, without using expensive fluorescent-dye detection devices.

### BACKGROUND OF THE INVENTION

A polymerase chain reaction method (hereinafter, also denoted simply as PCR method) is generally the technology implemented for amplifying genes. The PCR method was disclosed by Saiki (Science 230, 1350-1354 (1985)), and is a method in which, when a specific nucleotide sequence region (DNA in a specimen or the like) is to be detected, a DNA specimen in which one of the two ends of the specific region of a double-stranded DNA (one bing a + strand, and the other, a − strand) is identified as the + strand 3′ end and the other as the − strand 3′ end, and two oligonucleotides which will hybridize are prepared and are denatured by heat to form a single-stranded DNA, and this single-stranded DNA is caused to function as a primer in a template dependent nucleotide polymerization reaction, and the two DNA strands that are formed are once again separated into single strands, and by repeating the series of operations that cause the same reaction to recur, DNA is amplified until there is a detectable amount of the specified region which is interposed between the two primers

In a reaction system in which a primer is suitably designed, during the detection, the presence of only a small amount, for example, one virus, bacteria or cell, can be detected by assessing whether or not DNA has been amplified.

One method for checking the amplified product is the PCR-SSCP (PCR-Single Strand Conformation Polymorphism) method in which electrophoresis is carried out directly on the amplified product using a polyacryl amide, and mutations can be detected based on the difference in degree of electrophoretic migration.

In this method, the change in the three dimensional structure of the amplified product due to mutation is detected as a difference in the degree of electrophoretic migration. In addition to this, there is also sequence technology such as the shotgun method, the primer walking method and the cloning method; as well as the PCR-ASP (PCR-Allelle Specific Primer) method in which the DNA is suitably cut by a restriction enzyme and a PCR primer 3′ end is deliberately placed at the portion of mutation, and discrimination is achieved based on whether or not PCR is carried out; a PCR-RFLP (PCR-Restriction Fragmentation Length Polymorphism) method in which the mutated portion is amplified by PCR, and a distinction is made based on whether cutting can be done by a restriction enzyme; and the Tackman probe method in which the altered portion is also detected by a fluorescent marker probe. However, in each of these methods persons who are skilled in the high technology use expensive equipments and the cost in terms of time and finances is also high, but effective methods other than these have not been developed at the present time.

DNA hybridization uses hydrogen bonding of adenine (A) with thymine (T) and that of cytosine (C) and guanine (G), and there are 2 A-T hydrogen bonds, and 3 G-C hydrogen bonds, and at the portion where there are many G-C sequences, even if the A or T sequence is only slightly offset, in the prior art, they may be mistakenly hybridized. Consequently, this is not sufficiently accurate for definite diagnoses.

Meanwhile, a gene diagnosis method is proposed in the patent application of Maeda et al. (JP-A No. 2001-252098, in which the term, JP-A refers to an unexamined Japanese Patent Application), in which gene DNA is added to an aqueous solution including a DNA conjugate substance which is a complex of a single-stranded DNA and a hydrophobic substance and metallic anions, and the change in either the intensity of light scattering or light transmittance of the aqueous solution is measured. This is a method which utilizes the fact that the water solubility of the DNA is high and it forms a colloid, but when there is a double strand, and adenine and cytosine form hydrogen bonds with thymine and guanine, respectively, their hydrophilic properties decrease, and the colloid only becomes unstable causing aggregation when there is DNA that forms a completely complementary strand that is the same length of the DNA and thus hybridization occurs. This is a highly accurate detection method in which mutation of just one DNA such as the monobasic polymorph (SNPS) can be detected.

However, in order for this detection to be used in various genetic tests, large amounts of the specimen DNA must be collected, or the specimen DNA must be amplified and the obtained DNA must be cut using an expensive restrictive enzyme that is the same length as the DNA conjugate, and the other DNA which hinders hybridization of the DNA conjugate and the DNA must be removed, thereby labor-intensive and time-consuming processes had to be carried out. An improved method was sought in which the specimen could be used in an actual diagnosis without being adjusted.

### SUMMARY OF THE INVENTION

The present invention was conceived in view of the above situation, and the object thereof is to provide a simple method for DNA detection, and more specifically to provide a method for simply detecting whether the specimen DNA is the target DNA.

Thus, in one aspect the invention is directed to a DNA amplification method comprising:
(a) a denaturing step in which a double-stranded DNA is separated into two single-stranded DNA strands,
(b) an annealing step in which the two types of oligonucleotides (hereinafter, also referred to as primer) which are designed so that the end of the 3′-side of the respective region of the sequence to be amplified are identified and hybridized, is allowed to be hybridized to each single-stranded DNA that has been separated,
(c) a polymrase step in which oligonucleotides from DNA polymerase are subjected to an elongation reaction in the presence of deoxyribonucleotide triphosphate (dNTP), and
(d) repeating the foregoing (a) to (c) steps to perform DNA amplification to obtain a DNA complex,
   wherein in step (b), the two types of oligonucleotides are oligonucleotides in which a polymer having a lower critical solution temperature is linked to the end of the 5′-side.

In another aspect the invention is directed to an aggregate which is formed by a process comprising (i) adding an aqueous solution containing a metal ion to a composition containing a DNA obtained by a method as claimed in claim 1 and (ii) heating the composition at a temperature higher than the lower critical solution temperature of the polymer to cause the DNA to aggregate.

According to the present invention, a simple detection as to whether the target gene is present or not, can be done visually without expensive fluorescent reagents or fluorescent detection devices.

### DETAILED DESCRIPTION OF THE INVENTION

Firstly, the principle of the invention will be described.

The oligonucleotide used in the present invention in which polymers having a lower critical solution temperature are linked at the 5′ end (hereinafter, also referred to as oligonucleotide copolymer), is in a state in which all of the components are extended in a chain at temperatures below the transition temperature of the polymer that is linked to the oligonucleotide and the amplified DNA which will be the template (referred to hereinafter as template DNA or simply template) has a structure that is easily hybridized. In the case where poly (N-isopropylacrylamide) is selected as the polymer having a lower critical solution temperature, because the lower critical solution temperature of poly (N-isopropylacrylamide) is 32° C., it is annealed to the template at a temperature where hybridization with the template DNA easily occurs, for example, at about 25° C. After completing the annealing, the oligonucleotide is elongated along the template DNA using a polymerase enzyme. Because the polymerase enzyme generally used elongates the DNA at a temperature above 55° C., the temperature is above the transition temperature of the polymer having a lower critical solution temperature at the 5′ end, and a micelle structure is formed in which this portion is hydrophobic while the oligonucleotide is the hydrophobic region. Whether a micelle structure is formed or not depends on the structure of the synthesized DNA. That is to say, the length and the amount of the DNA in the synthesized DNA are important.

In the amplification method, when the annealing is done at a low temperature, there are some cases where abnormal amplification occurs due to mismatching. However, the mismatched portion is eliminated when this-type of micelle structure is formed, only normal amplification proceeds, and the DNA elongates to the portion where the other primer is annealed. As a result, DNA is synthesized in which a polymer having a lower critical solution temperature is linked to the 5′ end and which has a strand that is completely complementary to the template DNA.

The success of the DNA amplification is determined by using a temperature below the lower critical solution temperature, and by whether DNA in which a polymer having a lower critical solution temperature is linked to the 5′ end aggregate by increasing the salt concentration.

Next, the constituent elements will be described in order.

The oligonucleotide copolymer used in the annealing step contains a polymer exhibiting a lower critical solution temperature at the 5′ end of the oligonucleotide.

The lower critical solution temperature refers to the critical temperature at which the polymer changes from a state of being soluble in water to a state of being insoluble in water. At a temperature lower than this temperature, the polymer molecule is hydrated and the molecule chain is expanded, while at a temperature higher than this temperature, dehydration occurs and the molecular chain aggregates. These changes which are due to temperature are reversible.

The lower critical solution temperature may be varied by using at least one monomer that forms a polymer that does not exhibit a lower critical solution temperature, together with at least one monomer that can form a polymer that does exhibit a lower critical solution temperature.

In the case where a hydrophobic monomer, for example, is used as the monomer that forms the polymer that does not exhibit a lower critical solution temperature, the lower critical solution temperature can be shifted to the lower temperature side, while the use of a hydrophilic monomer shifts the lower critical solution temperature moves to the higher temperature side.

Examples of monomers that form the polymer that exhibits a lower critical solution temperature include an N-substituted (metha)acrylamide derivative, vinyl methyl ether and the like, and of these, the N-substituted (metha)acrylamide derivative is preferred.

The lower critical solution temperatures of the polymers that are obtained by polymerizing the monomers are, for example:
poly(N-ethylacrylamide): 72° C.
Poly(N-ethyl-N-methylacrylamide): 56° C.
poly(N-pyrrolidinylacrylamide): 56° C.
poly(N-cylopropylacrylamide): 46° C.
poly(N-isopropylacrylamide): 32° C.
poly(N-diethylacrylamide): 29° C.
poly(N-proplylacrylamide): 21° C.
poly(N-methyl-N-isopropylacrylamide): 20° C.
poly(N-piperidinylacrylamide): 5° C.
poly(N-cyclopropylmetacrylamide): 59° C.
poly(N-ethylmetacrylamide): 50° C.
poly(N-isopropylmetacrylamide): 44° C.
poly(N-propylacrylamide): 13° C.

Of these, poly (N-isopropylacrylamide) (also referred to as PNIPAAm hereinafter) is well known with respect to temperature response behavior (Schild, H. G., Prog. Polym. Sci. 17, 163-249, (1992)), and at the time when a complex is formed with a single-stranded DNA, the response temperature region is relatively low, and it is easily handlable and easily available (for example, from Kohjin Co., Ltd.), PNIPAAm is preferably used as the polymer exhibiting a lower critical solution temperature.

Other monomers may be polymerized with the monomers listed above as long as the lower critical solution temperature is not lost.

In the case where PNIPAAm is used as the polymer having a lower critical solution temperature which is linked to the 5′ end of the oligonucleotide of the oligonucleotide copolymer and the oligonucleotide is a single-stranded DNA, the oligonucleotide copolymer will be a single-stranded DNA-PNIPAAm. There are many suitable methods for producing this single-stranded DNA-PNIPAAm, but a favorable method is one in which an end-aminated single-stranded DNA and acryloyloxysuccinimide (obtained from Across Co., Ltd.) undergo a coupling reaction and a single-stranded DNA in which the end amino group is caused to be acryloyl is formed, and then N-isopropylacrylamide (NIPAAm hereinafter) monomer is radical-copolymerized to thereby obtain a single-stranded DNA-PNIPAAm. Any catalyst used for the coupling reaction and copolymerization reaction may be applicable, and the reaction conditions are not specifically limited. For example, the end-aminated single-stranded DNA and the acryloyloxysuccinimide may be coupled in a reaction system where the pH is adjusted in the presence of Na₂CO₃—NaHCO₃, and the pH of the product of the reaction and NIPAAm may be adjusted in the presence of Tris-HCl which is a buffer, and radical polymerization may be done with N, N, N′, N′-tetra methylethylenediamine as a surfactant, and ammonium persulfate or the like as the initiator.

It is to be noted that, methods for synthesizing the aminated end single-stranded DNA are described in detail in JP-A Nos. 59-27900, 59-93098, 59-93099 and 59-93100. In addition, the method for end vinylizing and a method for copolymerizing the vinylized end and NIPAAm are described in detail in papers by Maeda et. al (Biotechnology and Bioengineering Vol. 72, No. 2, 261-268 and Polymer Journal Vol. 33, No. 10, 830-833).

It is preferable that both of the two types of oligonucleotides used in the annealing step are oligonucleotide copolymers, but only one of them may be an oligonucleotide copolymer.

The oligonucleotide portion of the oligonucleotide copolymer used in the annealing step has a base sequence which is substantially complementary with a portion of the template DNA, and is hybridized at the respective 3′ side ends of the sequence region for amplification of the denatured (+) side DNA and (−) side DNA. Also the DNA strand is elongated after it is hybridized. It is to be noted that “base sequence which is substantially complementary” refers to a base sequence that can be annealed to the template DNA under the reaction conditions for use without mismatching.

The oligonucleotide portion of the oligonucleotide copolymer may be selected from a deoxyribonucleotide and an analog thereof (both together also referred to as deoxyribonucleotide). Examples of the deoxyribonucleotide do not only include unmodified ones but also includes modified ones. The modified deoxyribonucleotide used is not specifically limited and includes, for example, a (α-S) deoxyribonucleotide in which an oxygen atom which is linked to a phosphorous group is replaced by a sulfur atom, or a deoxyribonucleotide in which the hydroxy group at the 2-position of the ribose is replaced by a methoxy group.

In this specification, deoxyribonucleotide refers to a nucleotide in which the sugar residue is comprised of D-2-deoxyribose, and has one of adenine (A), cyotosine (C), guanine (G) and thymine (T), at the base portion (or at the 1-position). In addition, examples of the deoxyribonucleotide includes a deoxyribonucleotide which contains modified bases such as 7-deazaguanosine and deoxyribonucleotide analogs like deoxyribonucleotides.

Using a nucleotide analog as the oligonucleotide portion of the oligonucleotide copolymer is effective in view of control of higher-order structure formation and stability of annealing with the template. These oligonucleotide portions are synthesized, for example, by the phosphoamidite method using the DNA synthesizer model 394 manufactured by Applied Biosystems Inc. Other methods include the triester method, the H-phosphonate method, the thionate method and the like, and oligonucleotide portions synthesized by any of the methods may be used.

The polymerase step of the present invention will be described next. In this step, DNA which is to be targeted in the detection is elongated.

The polymerase step is performed by elongating a primer that has been hybridized with DNA (oligonucleotide) using DNA polymerase [deoxyribonucleotide triphosphate (dNTP)].

The deoxyribonucleotide triphosphate (dNTP) elongates the oligonucleotide, but deoxyribonucleotide triphosphate (dNTP) used in the PCR method and the like, that is, mixtures of DATP, dCTP, dGTP, dTTP may be favorably used as the deoxyribonucleotide triphosphate (dNTP).

Examples of the deoxyribonucleotide triphosphate (dNTP) include deoxyribonucleotide triphosphate (dNTP) analogs such as 7-deaza-dGTP, dITP and other nucleotide triphosphates provided that they are substrates for the DNA polymerase used. In addition, examples include derivatives of deoxyribonucleotide triphosphate (dNTP) and deoxyribonucleotide triphosphate (dNTP) analogs, and examples of these derivatives include derivatives that have a functional group, such as an amino-derivative.

The deoxyribonucleotide triphosphate (dNTP) may be prepared similarly to a conventional synthesis method, for example, using a DNA synthesizer.

The amount of the deoxyribonucleotide triphosphate (dNTP) and the oligonucleotide copolymer used in this invention is adjusted based on the base sequence of the DNA to be detected, the length of the fragment to be amplified and the structure of the reaction system, and, for example, the amount of the amplified product may be used as a guideline for preparation, but there is no specific limitation.

The DNA polymerase refers to an enzyme which synthesizes a new DNA strand using a DNA strand as a template and in addition to natural DNA polymerases, examples include mutated enzymes which have the above described activity. It is preferably a DNA polymerase which does not have 5′→3′ exonuclease activity.

The DNA polymerase usable in this invention is not specifically limited, and examples thereof include mutants of thermophilic bacillus family bacterial DNA polymerase which have lost 5′→3′ exonuclease activity such as Bacillus caldotenax (hereinafter also denoted simply as B. ca) or Bacillus stearothermophilus (hereinafter also denoted simply as B. st), and a large fragment of DNA polymerase 1 (Klenow fragment) originated from Escherichia Coli (hereinafter E. coli). In addition, any DNA polymerase, from room temperature to high temperature resistant ones, may be favorably used. B. ca is a thermophilic bacteria whose incubation temperature is ca. 70° C., and the bacterial B. ca DNA polymerase is known to have DNA-dependent DNA polymerase activity, RNA-dependent DNA polymerase activity(reverse transcription activity), 5′→3′ exonuclease activity, and 3′→5′ exonuclease activity. The foregoing enzymes may be purified from the source and thereby obtained or may be a recombined protein created by genetic engineering. In addition, the enzyme may be one which is modified by genetic engineering or other means through substitution, deletion, addition, insertion and the like, and examples include B. ca BEST DNA polymerase (manufactured by Takara Shuzo Co., Ltd.) which is B. ca that has lost 5′→3′ exonuclease activity.

Also, some DNA polymerases are known that have endonuclease activity, such as RNase H activity, under specific conditions. These DNA polymerases may be used in this invention. That is to say, examples include the aspects in which the DNA is used under conditions where the polymerase has RNase H activity, such as in the presence of Mn²⁺. In this aspect, the present invention can be achieved without adding the RNase H described above. That is to say, the B. ca DNA polymerase can have RNase H activity in a buffer that includes Mn²⁺. It is to be noted that the above aspect is not limited to B. ca DNA polymerase, and any known DNA polymerase that is known to have RNase H and other activity such as Tth DNA polymerase originating from Thermus thermophilus may be used in this invention.

In the method of this invention, when there is the possibility that the activity of the enzyme used may be reduced during the reaction, more enzyme may be added during the reaction. The same enzyme that was added to the reaction liquid at the start of the reaction may be used, or a different type of enzyme that has the same effects may be used. That is to say, providing that by adding the enzyme during the reaction, the detection sensitivity increases or the effect of increasing the amplified product is obtained, the type and properties of the enzyme is not specifically limited.

According to the DNA amplification, not only double-stranded DNA, but also a single-stranded DNA can be amplified, and the RNA nucleotide sequence may also be amplified using cDNA obtained by reverse transcription with RNA as a template.

The DNA and RNA usable in the embodiments of this invention may be prepared from various specimens that may include the said DNA and RNA.

The DNA amplification reaction and the detection method may also directly use the above-described specimens.

The specimens that contain DNA and RNA are not specifically limited, but examples thereof include living source specimens such as whole blood, serum, buffy coat, urine, feces, spinal, fluid, semen, saliva, tissue (such as cancer tissue and lymph nodes), cell cultures (such as mammalian cell cultures and bacterial cell cultures); specimens including animal and plant DNA such as viroids, viruses, bacteria, fungus, yeast; specimens (food and biological preparations) which may be mixed with or infected with microorganisms such as virus and bacteria; and specimens which may include organisms such as soil and waste water. Also, DNA or RNA preparation obtained by a processing the specimen using a known method may be used.

The preparation may, for example, be a cell fragment or a specimen obtained by dividing the cell fragment, DNA in the sample, or specific DNA molecule groups such as a mRNA enriched specimen may be used. In addition, the DNA or RNA in a specimen which has been amplified by a commonly known method may also be used.

Methods of preparing the preparation from the specimen is not specifically limited, and examples include dissolution using a surfactant; supersonic wave processing; agitation by shaking with glass beads, or a method using a French press. In a number of examples, (such as when an intrinsic nuclease exists), it is advantageous to use an additional operation to purify the DNA. In these examples, the purification is performed by known methods such as phenol extraction, chromatography, ion exchange, gel electrophoresis, or density gradient centrifugal separation.

The RNA used to form the DNA that has a sequence originating from the RNA is not specifically limited provided that it can produce a primer for use in the reverse transcription reaction, and can be all RNA in samples, and examples thereof include RNA molecule groups such as mRNA, tRNA, rRNA, or specific types of RNA molecules.

The primer for use in the reverse transcription reaction is not specifically limited, provided that it anneals to the template RNA under the used reaction conditions. The primer may be one having a base sequence which is complementary to the specific template RNA, an oligo-DT (deoxythymine) primer, or a primer that has a random sequence (random primer). The length of the primer for reverse transcription is preferably at least 6 nucleotides, and more preferably at least 9 nucleotides in view of carrying out special annealing, and 100 or less nucleotides and more preferably 30 or less nucleotides in view of synthesis of the oligonucleotides.

The enzyme for use in the reverse transcription reaction is specifically limited, provided that it has cDNA synthesis activity which will use RNA as a template, and examples thereof include various source reverse transcription enzymes such as avian myeloblastosis virus (AMV RTase), Moloney murine leukemia virus(MMLV RTase), Rous-associated virus (RAV-2 RTase) and the like. In addition, DNA polymerase which has reverse transcription activity along with other activity may be used. Enzymes which have reverse transcription activity at a high temperature are favorable, and examples include Thermus family bacterial Tth DNA polymerase (Tth DNA polymerase etc.) and Thermophilic bacillus family bacterial DNA polymerase. These are not specifically limited, but Thermophilic bacillus family bacterial DNA polymerase is preferable, as well as DNA polymerase from B. st (B. st DNA polymerase) and (B. ca DNA polymerase). B. ca DNA polymerase, for example, does not need any manganese ion for the reverse transcription reaction, and cDNA synthesis can be performed under high temperature conditions while controlling second order structure formation of the RNA template.

The enzyme which exhibits reverse transcription activity may use any one which is natural or modified provided that it has the above-mentioned activity.

In one aspect of this invention, DNA or RNA with the base sequence that is to be amplified may be replicated in advance, and used as the template RNA or forming the template DNA according to the method of this invention.

The method for DNA replication is not specifically limited, and one example thereof is a method in which after transformation is accomplished for a suitable host in a vector into which a DNA fragment having the base sequence which is to be amplified, the obtained transformant is cultivated, and the vector into which the DNA fragment having the base sequence which is to be amplified is inserted, is extracted and used. The vector is not specifically limited provided that it can be stably replicated witin the host. Examples of favorably used vectors are pUC, pBluescript, PGEM, cosmid, and fuzzy type vectors. Also, the host is not specifically limited provided that it can hold the vector, and examples thereof include E. coli bacteria and the like, that are easily cultivated. Further, another aspect of the replication method is one in which the DNA fragment having the base sequence which is to be amplified is used as a template to create a number of RNA having the said base sequence using RNA polymerase, and then a number of cDNA are formed by the reverse transcription reaction.

RNA replication can be performed using the DNA fragment having the base sequence which is to be amplified. The DNA fragment is not specifically limited providing that it has a RNA polymerase promoter sequence, and it may also be one which is inserted into a vector that has a RNA polymerase promoter sequence, or one in an adapter or cassette that has an endless RNA polymerase promoter sequence is ligated, or one which undergoes enzymatic synthesis using a primer that has a RNA polymerase promoter sequence and a suitable template. That is to say, the DNA fragment having the base sequence which is to be amplified can be replicated as RNA and amplified using the RNA polymerase promoter sequence that is arranged as described above. The vector is not specifically limited, providing that it has a RNA polymerase promoter sequence, and pUC, pBluescript, pGEM, cosmid, and fuzzy type vectors may be used. Also, the vector is preferably either one which retains its ring-shape or one which is processed to exhibit a straight chain. In addition, the RNA polymerase used in the above replication and amplification methods is not specifically limited, and SP6 RNA polymerase, T7 RNA polymerase, T3 RNA polymerase and the like may be favorably used.

Any double-stranded DNA such as a DNA genome which was isolated by the foregoing method or the PCR fragment, or single-stranded DNA such as the cDNA which was prepared from whole RNA or mRNA by reverse transcription reaction, may be favorably used as the template DNA in the amplification method of this invention.

In the case where DNA having the sequence derived from the RNA is to be amplified, the RNA strand of the RNA-cDNA double-stranded DNA which was obtained by the reverse transcription reaction using RNA as a template may be broken up using RNase H and the like, and amplified as a single-stranded cDNA. However, adding a RNA splitting enzyme such as RNase H to the reaction solution for amplification in this invention causes the amplifying reaction to start without splitting the RNA strand in advance. In addition, by using a DNA polymerase which has reverse transcription enzyme activity and chain substitution activity in the DNA amplification method of this invention, the reverse transcription reaction with RNA as a template and the DNA amplification reaction using the cDNA produced by this reaction as a template, are carried out using a single type of DNA polymerase.

The length of the template DNA must be such that the target sequence can be completely included in the fragment, or such that at least a sufficient portion of the target sequence is present in the fragment, and thus sufficient bonding of the primer sequence is provided.

The method of this invention is not specifically limited, but in the case where the template DNA is a double-stranded DNA, they are denatured to a single strand, and thus bonding of the oligonucleotide to the template DNA strand becomes possible. A method for denaturing is preferable in which the double-stranded DNA is maintained at a denaturing temperature, for example, at 95° C. Another method involves increasing the pH, but in order for the oligonucleotide to bond to the target, it is necessary to reduce the pH at the time of the amplification reaction. After the foregoing step in which the double-stranded DNA is denatured to form to a single-stranded DNA, or in the case where the template is RNA, after the step in which the cDNA (single-stranded DNA) is prepared by the reverse transcription reaction, the temperature is changed to thereby cause the DNA to be amplified. The temperature change may, for example, refer to annealing at 25° C., amplifying the DNA at 55° C., modifying the DNA at 95° C., and repeating this process continuously amplifies DNA.

The DNA amplification reaction of the invention can be carried out at normal temperature (example 37° C.) by using a normal temperature DNA polymerase such as the Klenow fragment, but by using a heat resistant enzyme (endonuclease, DNA polymerase) the amplification reaction can be carried out at high temperatures above 50° C. and even above 60° C.

In one embodiment of the amplification reaction, the reverse transcription reaction and the DNA amplification are carried out consecutively, and when a reverse transcription enzyme is used in this reaction, or when a DNA polymerase which has reverse transcription activity is used, DNA which has the original RNA sequence can be amplified.

The DNA polymerase which is used contributes to elongated strand synthesis from the 3′ end of the nucleotide portion in the downstream direction, and it is important that it does not have 5′→3′ exonuclease activity which may break up the substituted chain. Examples of this type of DNA polymerase include the Klenow fragment which is an exonuclease deletion transformant of DNA polymerase 1 from E coli and, a similar fragment from B. st DNA polymerase (manufactured by New England Biolabs) and B. ca BEST DNA polymerase from the B. ca (manufactured by Takara Shuzo Co., Ltd.). Sequence 1.0 and sequence 2.0 (manufactured by U.S Biochemicals), and T5DNA polymerase and φ29 DNA polymerase which are described in “Gene” vol. 97, pages 13-19 (1991), may also be used. Even for DNA polymerase which normally exhibits 5′→3′ exonuclease activity, the DNA synthesis method of this invention can be used when the activity can be inhibited by adding a suitable inhibitor.

In this invention, a reacting device such as an automatic thermal cycler may be used to perform amplification and the amplification is carried out by sequentially inserting the reaction device into a heat block in which the temperature is adjusted for the annealing step, the polymerase step, and the modification step.

It is preferable that the polymerase step of this invention is carried out at a suitable temperature for suitably maintaining the activity of the used enzyme. The reaction temperature depends on the used enzyme, but is preferably approximately 20° C. to approximately 80° C., more preferably 30° C. to 75° C., and a temperature of 50° C. to 70° C. is specifically preferred.

In the invention the reaction temperature is adjusted in accordance with the amount of GC in the template DNA and the amplification efficiency is thereby improved. The temperature at which the polymerase step is carried out depends on the elongated template length or the Tm value of the oligonucleotide, but for example, if the amount of GC in the template DNA is low, the polymerase step is carried out at 50 to 55° C.

When B. ca BEST DNA polymerase is, for example, used as the DNA polymerase which has reverse transcription activity, amplification of the DNA from the RNA which includes the step of preparing the cDNA from RNA (reverse transcription reaction) can be simply carried out using only one enzyme. In addition, the step of preparing the cDNA from RNA can be carried out independently, and the product (cDNA) can also be used as the template DNA in the method of this invention.

The DNA amplification method of this invention may be used in various experimental operations which use DNA amplification, such as DNA detection, marking and base sequence determination.

In addition, the DNA amplification method of this invention may be used as in-situ DNA amplification, DNA amplification on a solid phase substrate such as a DNA chip, or multiplex DNA amplification in which multiple regions are amplified simultaneously.

The melting temperature (also denoted as Tm) in this invention refers to the temperature at which the 2 strands of DNA are thermally denatured to form a single strand, and the Tm can be calculated using the formula below, as defined in “Current Protocols in Molecular Biology”:
Tm(° C.)=81.5+16.6*log[S]+0.41*(% GC)−(500/n)
[S]: salt mole concentration,
(% GC): GC amount in the oligoDNA (%),
n: length of the oligoDNA (bp),
provided that the [S] refers to [(sodium ion concentration)+(calcium ion concentration)+(Tris ion concentration)×0.67], and the magnesium ion concentration is not included in the calculation.

In the DNA detection method of this invention, the difference in the base sequence on the target DNA can be identified.

In this aspect, the primer is designed so that the middle portion of the primer is to be at the position of the specific base which attempts to identify the targeted base sequence, for example, a hydrogen bond is formed between the base and a base at the middle portion of the primer. In the case where this type of primer is used to carry out the amplification reaction, the amplification does not occur when there is a mismatch between the base sequence of the middle portion of the primer and that of the DNA used as the template, and thus there is no formation of the amplification product.

In the method of this invention, information can be obtained about a specific base on a gene as in the case of point mutation and single base exchange (single nucleotide polymorphysm, SNP).

Detection of the target DNA using the method of this invention may be done directly using a specimen that includes the DNA, or may be done by first amplifying the target DNA. In this case, the strand length of the target DNA to be amplified is not specifically limited, but in view of detecting the target DNA with high sensitivity, it is effective for the length to be in the region of 200 bp or less, and more preferably 150 bp or less.

In addition, in the detection method of this invention, by using a reaction buffer containing buffer components such as vicine, tricine, HEPES, phosphate, or tris, and an annealing solution containing spermidine or propylene diamine, the target DNA can be detected with even higher sensitivity from very small amounts of DNA specimens. In this case, the DNA polymerase used is not specifically limited.

In the case where gene diagnosis is conducted using the DNA detection method of this invention, the content of single-stranded DNA in the amplifying solution obtained by the DNA amplification method of this invention is not specifically limited, provided that stable micelle structures can be formed. If the content is too low, not only is it difficult to confirm changes in the intensity of light dispersion due to addition of complementary genes, but also micelles may not be formed, which is of course not favorable. In addition, an excessively high content of the single-stranded DNA included is not favorable, because it becomes difficult for the DNA that posesses the polymerase (DNA copolymer) to form micelles, and even if the micelles are formed, they are often unstable. Accordingly, it is preferable that the content is not less than about 0.1 mol % and not more than 0.5 mol %. However, since it is preferable that the content be changed in accordance with the length of the single-stranded DNA and the structure of the hydrophobic substance, the content is not limited to the range described above.

In the gene diagnosis using the DNA detection method of this invention, salts, for example, metal ions such as magnesium or sodium are allowed to be present together in the amplification solution after DNA amplification is completed.

It is known that in an ionic surfactant micelle and a polymeric micelle, when the counter-ion concentration in the solution is increased, a decrease in the critical micelle concentration (CMC) and an increase in the number of conjugates occur. That is to say, when the counter-ion increases, micelle formation can be achieved even with a small amount of surfactant, and the micelle particle diameter increases.

When gene diagnosis is done using the DNA detection method of the present invention, it is preferable that the metallic ions are also included, since, even a low concentration of the formed single-stranded DNA having a polymer (single-stranded DNA copolymer substance) results in micelle formation and an increased micelle particle size, thereby simplifying the measurement of light dispersion.

The metallic ion used here is not specifically limited, and examples thereof include magnesium ions and sodium ions. MgCl₂ and the like may be used as the magnesium ions and the concentration thereof is preferably 20 to 100 mM, and more preferably 30 to 50 mM. NaCl and the like may be used as the sodium ions, and the concentration thereof is preferably 0.1 to 2 M, and more preferably 0.5 to 1.5 M.

In the DNA detection method of this invention, it is preferable that, for a single-stranded DNA proportion in the range from 0.1 to 0.5 mol %, 30 mM of Mg²⁺ ions are added and then the temperature is raised to be above the phase transition temperature of the polymer that has the lower critical solution temperature (above 32° C. for PNIPAM). However, if the temperature is above the Tm of the amplified DNA, DNA and DNA hybridization become loose, and, for example, a temperature of 32 to 40° C. is preferable.

In the DNA detection method of this invention, other substances may be added to the DNA amplification solution in which amplification is completed, as long as formation of the double helix between the single-stranded DNA and its complementary strand, and measurement of the light dispersion intensity are not hindered.

### EXAMPLES

The invention will be further described based on examples but is by no mans limited to these examples.

### Example 1

Pseudomonas aeruginosa was coated by spraying it into an agar medium and cultivated therein for one night at 37° C.

DNA was extracted from the Pseudomonas aeruginosa using an I CAN GN Sample preparation kit (manufactured by Takara Bio), in accordance with the following procedure.
(1) 200 μL of Lysis solution was put into a 1.5 L microtube;
(2) One colony from the Pseudomonas aeruginosa which was cultivated was picked out with a toothpick and put into the microtube of the foregoing (1), and was properly dispersed in a vortex to obtain a dispersion;
(3) the thus obtained dispersion was put into a 37° C. heated block and maintained for 30 minutes;
(4) the dispersion was then subjected to heat processing in a 95° C. heated block for 5 minutes to thereby separate the double-stranded DNA into two single-stranded DNAs;
(5) this was set into a centrifugal separator which had been cooled to 5° C., and centrifugal separation was carried out for 5 minutes at 12,000 rpm, and the supernatant was collected.

Since the Pseudomonas aeruginosa was a bacterium derived from ceramidase, Pseudomonas aeruginosa is detected by detecting the ceramidase gene, and the sequence of this portion was checked using the genebank database.

```
      1 ggatcctctt cggcatctgg atgatggcgg tgcagtacat cgactatccg gcggacaacc
    61 acaagctcgg ctggaacgag atgctcgcct ggctgcgcag caagcgctgg gcgtgcatgg
  121 gtttcggcgg ggtgacctac ctggcgctgc tgatcccgct ggtcaacctg gtcatgatgc
  181 ccgccgccgt cgccggcgcc accctgttct gggtccgcga ggaaggcgag aaggcgctgg
  241 tgaaataagc atgcgcccgg tgtccgctgt cggcattgtc aggcgggcgt cagcgccctg
  301 acagccggcg tcgggcacac tacgaatgcc ctcgggagcg tcggcgcagg ccgcagccga
  361 gacctgcgcc agcccttgca gaccggctcg acgctgccga accgactggc cgccggtgcc
  421 tccccacgca ggcggccttt ttttacccgc ctgccttgcc gtccatgcag gatggccggc
  481 gctcaccccc ttcctgtccg ctacgccccc ctcgactcca ccaccctggc actacagtgg
  541 caaccaggcg aagcaggctc cggcccgctt tcgatgacca ccgtccccag cccgcccagc
  601 ggcggaaaac aagaagaggg tcgccatgtc acgttccgca ttcaccgcgc tcttgctgtc
  661 ctgcgtcctg ctggcgctct ccatgcctgc cagggccgac gacctgccct accgcttcgg
  721 cctgggcaag gtggacatca ccggcgaagc cgccgaagtc ggcatgatgg gttactcctc
  781 cctcgaacag aagaccgccg gcatccacat gcgccagtgg gcgcgtgcct tcgtgatcga
  841 ggaagcggcc agcggacgtc gcctggtcta cgtcaacacc gacctgggga tgaccttcca
  901 ggccgtgcac ctgaaggtcc tggcccggct caaggcgaag taccccggtg tctacgacga
  961 gaacaacgtg atgctcgccg ocaccoacac ccactccggt ccgggcggct totoccacta
1021 cgcgatgtac aacctgtcgg tqctcggctt ccaggaaaag accttcaacg ccatcgtcga
1081 cggcatcgtc cgctccatcg agcgqgccca ggccaggttg cagcccggcc gcctgttcta
1141 cggcagcggc gagctgcgca acgccagccg caaccgttcg ctgctgtcgc acctgaagaa
1201 tccggacatc gccggctacg aggatggcat cgacccgcag atgagcgtgc tcagcttcgt
1261 cgacgccaac ggcqagctgg ccggcgcgat cagttggttc ccggtgcaca gcacctcgat
1321 gaccaacgcc aatcacctga tctccccgga caacaagggc tacgcctcct atcactggga
1381 gcacgacgtc agccgcaaga gcggtttcgt cgccgccttc goccagacca atgccggcaa
1441 cctgtcgccc aacctgaacc tgaagcccgg ctccggtccc ttcgacaacg agttcgacaa
1501 cacccgcgag atcggtctgc gccaattcgc caaggcctac gagatcgccg gocaggocca
1561 ggaggaagtg ctcggcgaac tggattcgcg cttccgtttc gtcgacttca cccgcctgcc
1621 gatccgcccg gagttcaccg acggccagcc gcgccagttg tgcaccgcgg ccatcggcac
1681 cagcctggcc gccggtagca ccgaagacgg tccaggcccg ctggggctgg aggaaggcaa
1741 caatccgttc ctctcggccc ttggcgggtt gctcaccggc gtgccgccgc aggaactggt
1801 gcaatgccag gcggaaaaga ccatcctcgc cgacaccggc aacaagaaac cctacccctg
1861 gacgccgacg gtgctgccga tccagatgtt ccgcatcggc cagttggaac tgctcggcgc
1921 ccccgccgag ttcaccgtga tggccggggt gcggatccgc cgcgcggtgc aggcggccag
1981 cgaagcggcc ggtatccgcc atgtggtctt caatggctac gcgaatgcct atgccagcta
2041 cgtcaccacc cgcgaggaat acgccgccca ggaatacgaa ggcggctcga ccctctacgg
2101 cccctggacc caggccgcct accagctgtt gttcgtcgac atggcggtgg cgctgcgcga
2161 acgcctgccg gtggaaacct cggcgatagc gccggacctg tcctgctgcc agatgaactt
2221 ccagaccgga gtagtggccg atgatcccta tatcggcaag tccttcggcg acgtgttgca
2281 acaacccagg gaaagttatc gcatcggcga caaggtgacc gtcgctttcg tgaccggaca
2341 tccgaagaat gacttgcgca ccgagaagac tttcctggaa gtggtgaata tcggcaagga
2401 tggcaaacag acgcccgtga ccgttgccac cgataatgac tgggataccc aataccgctg
2461 ggagagagtg ggtatatctg cctcgaaagc gactatcagc tggtccattc caccagggac
2521 cgagcccggc cattactaca tcaggcacta tggcaacgcg aagaacttct ggacccagaa
2581 gatcagcgaa atcggcggct cgacccgctc cttcgaggtg ctcggcacca ctccctagcg
2641 ggctccagcc aaggtttcga gattcgccag ccaactttat gacgcatgaa agtcgtcaaa
2701 taaaatgtga tttaaaacac atgaacaagt gaccttttca ttca
```

Of the foregoing sequence, 456 bp within 1045-1500 were amplified, and the primers were set as follows:

| | |
|---|---|
| forward primer: | 5′-CGGCTTCCAGG-3′ |
| reverse primer: | 5′-TTGTCGAACTC-3′ |

The absorbance of the oligonucleotide (at 260 nm) is calculated using the following formula:
E₂₆₀=A*15300+C*7400+G*11800+T*9300

In the formula, A is the number of adenosines contained in the oligonucleotide, C is the number of cytosines contained in the oligonucleotide, G is the number of guanines contained in the oligonucleotide, and T is the number of thymines contained in the oligonucleotide.

Using the formula, the absorbance (EF) of the forward primer is:
EF=1*15300+4*7400+4*11800+2*9300=110700.
The absorbance (ER) of the reverse primer is:
ER=2*15300+3*7400+2*11800+4*9300=113600.

The forward primer and the reverse primer, and those which were each aminohexylated on the 5′ end position were obtained by ordering a company that synthesized oligoDNA (Sigma Genomics).

The forward primer of 0.6 μmol, which was aminohexylated on the 5′ end, was dissolved in 150 μL of a carbonate buffer comprised of Na₂CO₃) (10 mM) and NaHCO₃ (90 mM) to prepare a primer solution.

In 460 μL of dimethyl sulfoxide was dissolved 8.5 μmol of acryloyloxysuccinimide (manufactured by Across Co.), and the obtained solution was added to the foregoing primer solution and allowed to react at room temperature for 24 hours. The solution was added to 10 mL of isopropanol and, after being mixed well, was put in a centrifugal separator; the supernatant was removed and a vinyl group was introduced to the 5′ end.

Similarly, the 5′ end of the reverse primer which was aminohexylated at the 5′ end, was vinylated.

The forward primer with the vinylated end and the reverse end with the vinylated end are respectively radical-polymerized in the presence of N-isopropylacrylamide (NIPAAm) and tetramethylethylenediamine, using ammonium persulfate as an initiator to synthesize a forward primer-PNIPAAm complex and a reverse primer-PNIPAAm complex. The obtained reaction mixture was dialyzed and then subjected to gel filtration is done in a column that was filled with SHEPHADEX G100 to remove unreacted components.

The thus obtained polymeric product was subjected to freeze-drying, and then dissolved in 25 mL of purified water, and the absorbance at 260 nm was as follows:
solution obtained from the forward primer: 2.16 (called primer A solution hereinafter), and
solution obtained from the reverse primer: 2.29 (called primer B solution hereinafter).

Polymerase and PCR reaction buffer, and the dNTP mixture kit “Pyrobest DNA Polymerase” were obtained from Takara Bio.

The following composition was mixed in a 1.5 ml PCR tube, and the thermocycle of 10 seconds at 98° C., 2 minutes at 25° C., and 1 minute at 68° C., was repeated 40 times.

| | |
|---|---|
| Supernatant with DNA extracted | 100 μL |
| from Pseudomonas aeruginosa | |
| 10 × Pyrobest Buffer | 100 μL |
| dNTP mixture | 80 μL |
| Primer A solution | 253 μL |
| Primer B solution | 245 μL |
| Sterilized distilled water | 222 μL |

The obtained reaction mixture was heated at 40° C. which was above the lower critical solution temperature of 32° C. for the PNIPAM. The reaction solution was transparent, but when 100 μL of 330 mM magnesium chloride aqueous solution was added, the reaction solution became cloudy, and after 10 minutes the transmittance was less than 5%.

Similarly, salt was added to the reaction solution in which the thermal cycle was not conducted, and in view of the fact that it did not become cloudy even when heated to 40° C., it was concluded that the PCR reaction proceeded.

### Example 2

The sequence of a DNA (φX174 RF 1 DNA: plasmid gene, available from Wako Pure Chemical Industries) can be checked using the gene bank, and “ATTGCTGGCA” is used as the forward primer, and “ATTCTGGCGT” is used as the reverse primer, and the 36 bp between 3456 and 3491 was subjected to the PCR.

Synthesis of Forward Primer with Vinylated 5′ End

To 1 μmol (approximately 3.2 mg) of a forward primer with an aminated 5′ end was added a solution in which sodium carbonate and sodium bicarbonate were each dissolved in pure water in an amount of 100 mmol/l, and pure water was further added to make up 500 μL.

Acryloyloxysuccinimid of 8.46 mg was dissolved in 150 μL of dimethylsulfoxide and was added with mixing to the foregoing solution of the forward primer with an aminated 5′ end, and then allowed to react for 24 hours at room temperature. After completion of the reaction, the reaction solution was put into 10 mL of ethanol while vigorously mixing, and then the precipitate was collected using a centrifuge separator, washed 3 times in ethanol, and subsequently dried for 48 hours at 30° C. in a drying box. When the obtained powder was checked with reversed phase HPLC, there were observed no peaks for the forward primer with an aminated 5′ end, the acryloyloxysuccinimide or the decomposition product, and it was thus proved that a forward primer with a vinylated 5′ end had been obtained.

Bonding of Polymer to Forward Primer

A polymer exhibiting a lower critical solution temperature was allowed to bond to the 5′ end of a forward primer according to the following procedure. 100 mmol/L Tris-HCL aqueous solution (manufactured by Wako Pure Chemical Industries) was diluted by a factor of 10 with pure water to prepare 10 mmol/L Tris-HCl aqueous solution (hereinafter, also denoted simply as a buffer solution).

Ammonium persulfate of 74.1 mg was weighed and put into a 25 mL measuring flask and made up to 25 mL with the buffer solution (hereinafter, also denoted as an initiator solution).

N, N, N, N-tetramethylethylenediamine of 6.235 g was weighed, put into a 25 mL measuring flask and made up to 25 mL with the buffer solution (hereinafter, also called an additive solution); 1.75 mg (0.5 μmol) of the forward primer with a vinylated 5′ end and 15.82 mg (140 μmol) of NIPAN (isopropyl acrylamide, manufactured by Kohjin Co., Ltd which was re-crystallized with hexane) were dissolved. Initiator solution of 100 μL and 40 mL of the additive solution were added to this, and after nitrogen was bubbled into it, the mixture was allowed to react for 2 hours at 25° C.

The obtained reaction solution was dialyzed with a dialysis membrane (Spectra/Por6, MWCO =1000), and subjected to gel filtration using a Sephadex G-100 column to remove any unreacted products, whereby a polymer exhibiting a lower critical solution temperature was linked to the 5′ end of the forward primer.

The amount of the obtained polymer was 12.3 mg. Based on the absorbance of 260 nm of this polymer, it was proved that the content of the forward primer unit in all the polymer units was 0.35 mol %.

Subsequently, a solution having the composition as shown below was prepared. The preparation was accomplished at a low temperature while cooling the container with iced water. The obtained polymer of 12.3 mg was dissolved in 3.5 mL of sterilized water (Solution A).

Meanwhile, as a reverse primer, the oligoDNA of ATTCTGGCGT was dissolved in sterilized water so that a 0.1 mmol/L solution was formed (Solution B).

15 μg φX174 RF I DNA was dissolved in 750 μL of sterilized water (Solution C). Also, a Takara Pyrobest DNA Polymerase set was prepared.

Reagents were mixed in the PCR tube in the order below. After the necessary amounts of the reagents were mixed, they were mixed again by light pipetting.

| | |
|---|---|
| Sterilized water: | 29.5 μL |
| Solution C: | 50.0 μL |
| 10 × Pyrobest DNA Polymerase: | 0.5 μL |
| 10 × Pyrobest Buffer 2: | 10.0 μL |
| dNTP mixture: | 8.0 μL |
| Solution A: | 1.0 μL |
| Solution B: | 1.0 μL |

While the reagents were mixed, a 25° C. hot water bath, a 68° C. hot water bath, and a 98° C. oil bath were prepared, and the container in which the reagents were mixed was:
(a) immersed in the 98° C. oil bath for 1 minute,
(b) immersed in the 25° C. hot water bath for 1 minute,
(c) immersed in the 68° C. hot water bath for 30 seconds
(d) immersed in the 98° C. oil bath for 2 seconds, and further the immersions of the foregoing (b), (c) and (d) were repeated 30 times.

The obtained reaction solution was mixed with 100 μL of Mg²⁺ 60 mol/L in pure water, and when the container was heated to 40° C., it became cloudy and aggregates were generated.

Meanwhile, instead of the foregoing solution A, a solution in which ATTGCTGGCA oligoDNA of ATTGCTGGCA was dissolved in sterilized water and made up to 0.1 mmol/l, was added, and the procedure similar to the foregoing was carried out, but the aggregation did not occur.

The solutions which caused aggregation and those which did not cause aggregation were subjected to electrophoresis, together with 30 mer and 40 mer markers, whereby it was proved that PCR was successfully performed in both because there was a band between the markers in both cases.

1. A DNA amplification method comprising the steps of:
(a) denaturing a DNA to separate a double-stranded DNA into two single-stranded DNA,
(b) hybridizing two types of oligonucleotides which were designed so that the end of the 3′ side of the region of a sequence to be amplified is identified and hybridized with each single-stranded DNA that has been separated,
(c) elongating the oligonucleotides with a DNA polymerase in the presence of deoxyribonycleotide triphosphate, and
(d) repeating steps (a) through (c) to perform DNA amplification,
wherein in step (b), the two types of oligonucleotides are oligonucleotides in which a polymer having a lower critical solution temperature is linked to the 5′ end.

2. The method of claim 1, wherein the polymer is selected from the group consisting of poly(N-ethylacrylamide), Poly(N-ethyl-N-methylacrylamide), poly(N-pyrrolidinylacrylamide), poly(N-cylopropylacrylamide), poly(N-isopropylacrylamide), poly(N-diethylacrylamide), poly(N-methyl-N-isopropylacrylamide), poly(N-proplylacrylamide), poly(N-piperidinylacrylamide), poly(N-cyclopropylmetacrylamide), poly(N-ethylmetacrylamide), poly(N-isopropylmetacrylamide) and poly(N-propylacrylamide).

3. The method of claim 1, wherein the polymer is poly(N-isopropylacrylamide).

4. The method of claim 1, wherein the polymer has a lower critical solution temperature of 20° C. to 46° C..

5. The method of claim 1, wherein the method further comprises:
(e) adding an aqueous solution containing a metal ion to a composition containing an amplified DNA obtained in step (d) and then heating the composition at a temperature higher than the lower critical solution temperature of the polymer to form aggregates, and
(f) measuring a difference in scattered light intensity or light transmittance between before and after adding the aqueous solution.

6. The method of claim 5, wherein in step (e), heating is performed at a temperature lower than a melting temperature (Tₘ) of the double-stranded DNA.

7. An aggregate which is formed by a process comprising (i) adding an aqueous solution containing a metal ion to a composition containing a DNA obtained by a method as claimed in claim 1 and (ii) heating the composition at a temperature higher than the lower critical solution temperature of the polymer to cause the DNA to aggregate.

8. The aggregate of claim 7, wherein the metal ion is magnesium ion or sodium ion.

9. The aggregate of claim 8, wherein the aqueous solution contains the magnesium ion at a concentration of 20 to 100 mM.

10. The aggregate of claim 8, wherein the aqueous solution contains the sodium ion at a concentration of 0.1 to 2 M.

11. A DNA complex obtained by a method as claimed in claim 1.


SEQUENCE LISTING (5 sequences)

SEQ ID NO: 1 - DNA, 2744 bp, Pseudomonas aeruginosa
ggatcctcttcggcatctggatgatggcggtgcagtacatcgactatccggcggacaaccacaagctcggctggaacgagatgctcgcctggctgcgcagcaagcgctgggcgtgcatgggtttcggcggggtgacctacctggcgctgctgatcccgctggtcaacctggtcatgatgcccgccgccgtcgccggcgccaccctgttctgggtccgcgaggaaggcgagaaggcgctggtgaaataagcatgcgcccggtgtccgctgtcggcattgtcaggcgggcgtcagcgccctgacagccggcgtcgggcacactacgaatgccctcgggagcgtcggcgcaggccgcagccgagacctgcgccagcccttgcagaccggctcgacgctgccgaaccgactggccgccggtgcctccccacgcaggcggcctttttttacccgcctgccttgccgtccatgcaggatggccggcgctcacccccttcctgtccgctacgcccccctcgactccaccaccctggcactacagtggcaaccaggcgaagcaggctccggcccgctttcgatgaccaccgtccccagcccgcccagcggcggaaaacaagaagagggtcgccatgtcacgttccgcattcaccgcgctcttgctgtcctgcgtcctgctggcgctctccatgcctgccagggccgacgacctgccctaccgcttcggcctgggcaaggtggacatcaccggcgaagccgccgaagtcggcatgatgggttactcctccctcgaacagaagaccgccggcatccacatgcgccagtgggcgcgtgccttcgtgatcgaggaagcggccagcggacgtcgcctggtctacgtcaacaccgacctggggatgaccttccaggccgtgcacctgaaggtcctggcccggctcaaggcgaagtaccccggtgtctacgacgagaacaacgtgatgctcgccgccacccacacccactccggtccgggcggcttctcccactacgcgatgtacaacctgtcggtgctcggcttccaggaaaagaccttcaacgccatcgtcgacggcatcgtccgctccatcgagcgggcccaggccaggttgcagcccggccgcctgttctacggcagcggcgagctgcgcaacgccagccgcaaccgttcgctgctgtcgcacctgaagaatccggacatcgccggctacgaggatggcatcgacccgcagatgagcgtgctcagcttcgtcgacgccaacggcgagctggccggcgcgatcagttggttcccggtgcacagcacctcgatgaccaacgccaatcacctgatctccccggacaacaagggctacgcctcctatcactgggagcacgacgtcagccgcaagagcggtttcgtcgccgccttcgcccagaccaatgccggcaacctgtcgcccaacctgaacctgaagcccggctccggtcccttcgacaacgagttcgacaacacccgcgagatcggtctgcgccaattcgccaaggcctacgagatcgccggccaggcccaggaggaagtgctcggcgaactggattcgcgcttccgtttcgtcgacttcacccgcctgccgatccgcccggagttcaccgacggccagccgcgccagttgtgcaccgcggccatcggcaccagcctggccgccggtagcaccgaagacggtccaggcccgctggggctggaggaaggcaacaatccgttcctctcggcccttggcgggttgctcaccggcgtgccgccgcaggaactggtgcaatgccaggcggaaaagaccatcctcgccgacaccggcaacaagaaaccctacccctggacgccgacggtgctgccgatccagatgttccgcatcggccagttggaactgctcggcgcccccgccgagttcaccgtgatggccggggtgcggatccgccgcgcggtgcaggcggccagcgaagcggccggtatccgccatgtggtcttcaatggctacgcgaatgcctatgccagctacgtcaccacccgcgaggaatacgccgcccaggaatacgaaggcggctcgaccctctacggcccctggacccaggccgcctaccagctgttgttcgtcgacatggcggtggcgctgcgcgaacgcctgccggtggaaacctcggcgatagcgccggacctgtcctgctgccagatgaacttccagaccggagtagtggccgatgatccctatatcggcaagtccttcggcgacgtgttgcaacaacccagggaaagttatcgcatcggcgacaaggtgaccgtcgctttcgtgaccggacatccgaagaatgacttgcgcaccgagaagactttcctggaagtggtgaatatcggcaaggatggcaaacagacgcccgtgaccgttgccaccgataatgactgggatacccaataccgctgggagagagtgggtatatctgcctcgaaagcgactatcagctggtccattccaccagggaccgagcccggccattactacatcaggcactatggcaacgcgaagaacttctggacccagaagatcagcgaaatcggcggctcgacccgctccttcgaggtgctcggcaccactccctagcgggctccagccaaggtttcgagattcgccagccaactttatgacgcatgaaagtcgtcaaataaaatgtgatttaaaacacatgaacaagtgaccttttcattca

SEQ ID NO: 2 - DNA, 11 bp, Artificial Sequence
  Description of Artificial Sequence Primer
cggcttccagg

SEQ ID NO: 3 - DNA, 11 bp, Artificial Sequence
  Description of Artificial Sequence Primer
ttgtcgaactc

SEQ ID NO: 4 - DNA, 10 bp, Artificial Sequence
  Description of Artificial Sequence Primer
attgctggca

SEQ ID NO: 5 - DNA, 10 bp, Artificial Sequence
  Description of Artificial Sequence Primer
attctggcgt